(19) United States
(12) Patent Application Publication
(10) Pub. No.: US 2001/0007857 A1
(43) Pub. Date: Jul. 12, 2001

(54) METHOD FOR TREATING FELINE IMMUNODEFIENCY VIRUS INFECTIONS

(76) Inventor: SHALOM Z. HIRSCHMAN, RIVERDALE, NY (US)

Correspondence Address: MYRON COHEN, COHEN, PONTANI, LIEBERMAN & PAVANE, 551 FIFTH AVENUE, SUITE 1210, NEW YORK, NY, 10176

(21) Appl. No.: 08/835,796

(22) Filed: Apr. 15, 1997

(51) Int. Cl. A61K 38/00

(52) U.S. Cl. 514/12

(57) ABSTRACT

The present invention discloses a method for treating patients having the FIV associated symptoms or patients carrying or infected by the FIV or having antibodies against the FIV is disclosed using Product R, a peptide-nucleic acid preparation.

### BACKGROUND OF THE INVENTION

I. Field of the Invention

The present invention relates to a method for using Product R as hereinafter defined to treat patients infected with feline immunodeficiency virus (FIV).

II. Description of the Related Art

Treatment of viral diseases in humans is a major focus of medical science. While some progress has been made, viral infections are still among the diseases most difficult to treat. Despite growing understanding of viral diseases along with improved techniques for detecting and treating them, few antiviral drugs have proved effective. Some viral diseases such as HIV are life threatening; others such as herpes simplex virus and influenza virus continue to cause severe problems. Further, new viral diseases constantly appear as an inevitable consequence of evolution. Thus, searching for a novel and effective way of treating viral diseases remains imperative and challenging.

Product R¹ emerged as an antiviral product in the 1930's. While it was originally believed to be a product composed of peptone, peptides and nucleic acids (fully defined hereafter), the precise composition remains unidentified. Nevertheless, Product R has 1. The agent is known under the trademark “Reticulose”, a trademark of Advanced Viral Research Corp. demonstrated an ability to inhibit rapidly the course of several viral diseases. It is nontoxic, miscible with tissue fluids and blood sera and free from anaphylactogenic properties.

Despite these early promising clinical reports, systematic studies have rarely been performed to establish clinical utility. Optimum dosages of Product R for treating viral infections as indicated above have been poorly investigated. In fact, most of the clinical reports lacked necessary controls and statistically sufficient samples for evaluating the effectiveness of Product R. Note, two earlier publications challenged that Product R failed to demonstrate antiviral activity. In light of this controversy, the present status of the art of using Product R in treating viral infections remains questionable. Close examination of the development history of Product R reveals no meaningful pattern that could be followed to designate a treatment for a particular viral infection, for viruses causing those infections are extremely diversified in their genetic traits or/and pathogenesis. In addition, earlier clinical applications described Product R only as an agent to be administered alone. Product R has never been suggested to be applied in combination with other antiviral drugs; nor has Product R been administered for a period longer than about two months. Given the limits of prior art, developing new treatment strategies using Product R is desirable.

In developing an antiviral agent, it is well known that inhibitory activity of an antiviral agent against a particular virus cannot be equated with its inhibitory effect against another virus. For example, acyclovir has proved to be specifically effective against herpes simplex 1 and 2 but not against cytomegalovirus (CMV), even though both HSV and CMV belong to the same herpesvirus family, sharing certain genetic features. The specificity of acyclovir rests on the activity of the thymidine kinase gene unique to HSV 1 and 2, indicating that a distinctive feature of each individual virus forms a basis for developing an antiviral agent specifically against this very virus. In other words, treatment of a viral infection using a certain antiviral agent does not necessarily indicate that the same agent will produce the same effect when used for treating other viral infections. The genetic diversity of viruses further mandates that an attempt to be made to discern the effectiveness of a new application of an antiviral agent to a different virus.

An antiviral agent usually interacts with molecules involved in different stages of viral infections: in early events such as adsorption, penetration (internalization), and uncoating; in virus replication characteristic for each virus genome and components of the nucleoprotein complex; and in the chemistry of metabolic pathways. The best targets for inhibition by an antiviral agent are molecules serving a function unique to the virus, with no analogous counterpart in host cells. In order to identify the virus-specific molecule with which a putative antiviral agent interacts, it is important to characterize viruses in terms of particle and genome structure, as well as to define specific biochemical events that occur in infected cells. Although progress has been made in discovering molecules necessary for virus adsorption, replication and metabolism, current knowledge remains insufficient to explain many aspects of these events. Consequently, not every antiviral agent's function is fully defined in terms of its interaction with a target virus through one or a series of the indicated events; much less is understood where an antiviral agent is employed to treat a new viral infection, especially if the antiviral agent has been poorly characterized. Without the knowledge of a virus' genetic traits and the chemical properties of an antiviral agent, treatment of a viral infection becomes unpredictable.

Feline immunodeficiency virus (FIV) is a member of the retrovirus group which includes human immunodeficiency virus (HIV). Not closely related to HIV, FIV causes immunodeficiency diseases in cats. Limited serologic surveys suggest that FIV causes infection mainly in free-roaming cats and that the incidence is highest in older males. Because of the propensity for fighting among tom-cats, it is thought that biting is the major mechanism for spread. Infectious virus is present in saliva. In addition, gingivitis is relatively common among these cats, and inflammatory cells in the lesions may contain virus. Antibodies to FIV were also seen in several species of wild felids in zoos and in Florida panthers in the wild.

Infected cats develop fever, weight loss, lethargy, and lymphadenopathy. These are often accompanied by diarrhea, gingivitis, upper respiratory infections, and neoplastic tumors. Many animals develop anemia, lymphopenia, and neutropenia. Older animals may develop immunodeficiency, characterized by wasting and opportunistic infections. Kittens experimentally infected with FIV develop fever, neutropenia, and lymphadenopathy lasting several months. Animals then progress into a subclinical phase that may last for several years before the onset of terminal illness. FIV, like HIV can also produce encephalitis, with perivascular lymphocytic infiltrates.

Genomes of FIV, like HIV, are characterized by a complex combination of genes in addition to gag, pol and env. Similar to HIV, sequence analyses of several geographic isolates of FIV have revealed eight to nine variable regions within the FIV glycoprotein. A linear neutralization site has recently been identified in the V3 region of the FIV SU that has many structural features in common with the V3 of HIV, such as the presence of a Cys/Cys bond, an N-linked glycosylation site and a predicted-turn structure.

Insofar as the applicant knows, Product R has never been used, nor suggested for treating patients having virus infections other than human. It is now discovered that Product R is useful in treating animals having FIV infections.

### SUMMARY OF THE INVENTION

The object of this invention therefore is to provide a method for treating patients infected by feline immunodeficiency virus (FIV), or exhibiting FIV associated symptoms, or having antibodies against FIV, by administering parenterally to the patients Product R, an antiviral agent composed of peptides and nucleic acids.

More specifically, the present invention relates to a method for treating the identified patients by administering parenterally to the patients an effective FIV treatment amount of Product R from about 5 microliters to about 40 microliters per kilogram of body weight per day in a sterile injectable formulation.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims.

### DETAILED DESCRIPTION OF THE PRESENTLY PREFERRED EMBODIMENTS

As used herein, Product R is the product produced according to either of the following methods.

### Method I For Preparing Product R

Suspend about 35.0 g of casein, about 17.1 g of beef peptone, about 22.0 g of nucleic acid (RNA), about 3.25 g bovine serum albumin in about 2.5 liters of water for injection USP at about 3 to 7°C. in a suitable container and gently stir until all the ingredients have been properly wet. Carefully add while stirring about 16.5 g of sodium hydroxide (reagent grade ACS) and continue stirring until sodium hydroxide completely dissolved. Autoclave at about 9 lbs pressure and 200-230°F. for a period of time until RNA is completely digested, for example, about 4 hours. At the end of the period, the autoclave is stopped and the reaction flask and contents are permitted to slowly cool to ambient temperature. Then cool for at least six hours at about 3-8°C. The resulting solution is filtered through 2 micron and 0.45 micron filters using inert gas such as nitrogen or argon at low pressure (1-6 psi). In a similar manner the solution is filtered again through 0.2 micron pyrogen retention filters. The resulting filtrate is sampled and assayed for total nitrogen. A calculation is then performed to determine the quantity of cooled water for injection to be added to the filtrate to yield a diluted filtrate with a nitrogen content between about 165-210 mg/100 ml, the final volume is approximately 5 liters. The pH is then adjusted with either concentrated HCl (reagent grade ACS) or 1.0 normal NaOH to about 7.3-7.6 range. The diluted solution is then filtered again through 0.2 micron filters with inert gas at low pressure. The final filtrate is then filled and sealed into 2 ml glass ampules while in an inert gas atmosphere. The ampules are collected and autoclaved for final sterilization at 240°F. and 20 to 30 pounds pressure for about 30 minutes. Following the sterilization cycle, the ampules with Product R are cooled and washed.

All quantities are subject to plus or minus 2.5% variation for pH, volume, and analytical adjustments.

### Method II For Preparing Product R

Suspend about 35.0 g of casein, about 17.1 g of beef peptone, about 22.0 g of nucleic acid (RNA), about 3.25 g bovine serum albumin in about 2.5 liters of water for injection USP at about 3 to 7°C. in a suitable container and gently stir until all the ingredients have been properly wet. Slowly add while stirring about 11.75 ml of hydrochloric acid (reagent grade ACS) and continue stirring until hydrochloric acid is completely dissolved. Autoclave at about 9 lbs pressure and 200-230°F. for a period of time until RNA is completely digested, for example, about 4 hours. At the end of the period, the autoclave is stopped and the reaction flask and contents are permitted to slowly cool to ambient temperature. Then cool for at least six hours at about 3-8°C. The resulting solution is filtered through 2 micron and 0.45 micron filters using inert gas such as nitrogen or argon at low pressure (1-6 psi). In a similar manner the solution is filtered again through 0.2 micron pyrogen retention filters. The resulting filtrate is sampled and assayed for total nitrogen. A calculation is then performed to determine the quantity of cooled water for injection to be added to the filtrate to yield a diluted filtrate with a nitrogen content between about 165-210 mg/100 ml, the final volume is approximately 5 liters. The pH is then adjusted with either concentrated HCl (reagent grade ACS) or 35% (w/v) of NaOH to about 7.3-7.6 range. The diluted solution is then filtered again through 0.2 micron filters with inert gas at low pressure. The final filtrate is then filled and sealed into 2 ml glass ampules while in an inert gas atmosphere. The ampules are collected and autoclaved for final sterilization at 240°F. and 20 to 30 pounds pressure for about 30 minutes. Following the sterilization cycle, the ampules with Product R are cooled and washed.

All quantities are subject to plus or minus 2.5% variation for pH, volume, and analytical adjustments.

For patients with the above FIV infections or FIV associated symptoms, or having antibodies against FIV, a suitable effective dose of Product R will be in the range of from about 5 microliters to about 40 microliters per kilogram of body weight per day, preferably in the range of about 10 microliters to about 25 microliters per kilogram of body weight per day. Most preferably Product R is administered in an amount of about 30 microliters per kilogram of body weight per day for about one week, followed by about 15 microliters per kilogram of body weight per day in a sterile injectable formulation until the patient becomes asymptomatic or viral load becomes undetectable. The desired dose may be administered as two, three or more sub-doses at appropriate intervals, generally equally spread in time, throughout the day. Preferably, the full daily dose is administered in one administration.

Product R may be administered by any suitable injection route including, but not limited to intravenously, intraperitoneally, subcutaneously, intramuscularly, and intradermally, etc. The presently preferred route of administration is intramuscularly. It will be appreciated that the preferred route may vary with, for example, the condition and age of the recipient.

Product R may be used in therapy in conjunction with other medicaments including corticosteroid, gamma globulin, glucose, or vitamins, antiviral agents such as interferon or interleukin, etc..

While it is possible for Product R to be administered as part of a pharmaceutical formulation, it is preferable to present it alone, although it may be administered at about the same time as one or more other pharmaceuticals are independently administered. If Product R is administered as part of a pharmaceutical formulation, the formulations of the present invention comprise at least one administered ingredient, as above defined, together with one or more acceptable carriers thereof and optionally other therapeutic ingredients. The carrier(s) must be “acceptable” in the sense of being compatible with the other ingredients of the formulation and not deleterious to the recipient thereof. Preferably, Product R constitutes at least about 90% of such formulation by weight.

The formulations may conveniently be presented in unit-dose or multi-dose containers, e.g. sealed ampules and vials.

Preferred unit dosage formulations are those containing a daily dose or unit, daily sub-dose, or an appropriate fraction of the administered ingredient.

Thus, while there have been shown and described and pointed out fundamental novel features of the invention as applied to preferred embodiments thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. It is the intention, therefore, to be limited only as indicated by the scope of the claims appended hereto.

I claim:

1. A method of treating a patient having FIV associated symptoms, comprising administering parenterally to said patient an effective FIV treatment amount of Product R in a sterile injectable formulation.

2. The method of claim 1 in which an effective treatment amount of Product R is in a range from about 5 microliters to about 40 microliters per kilogram of body weight per day in a sterile injectable formulation.

3. The method of claim 1 in which an effective treatment amount of Product R is in a range from about 10 microliters to about 25 microliters per kilogram of body weight per day in a sterile injectable formulation.

4. The method of claim 1 in which an effective treatment amount of Product R is about 30 microliters per kilogram of body weight per day in a sterile injectable formulation for about one week, followed by about 15 microliters per kilogram of body weight per day in a sterile injectable formulation until said patient becomes asymptomatic or viral load becomes undetectable.

5. A method of treating a patient infected by or carrying the FIV, comprising administering parenterally to said patient an effective FIV treatment amount of Product R in a sterile injectable formulation.

6. The method of claim 5 in which an effective treatment amount of Product R is in a range from about 5 microliters to about 40 microliters per kilogram of body weight per day in a sterile injectable formulation.

7. The method of claim 5 in which an effective treatment amount of Product R is in a range from about 10 microliters to about 25 microliters per kilogram of body weight per day in a sterile injectable formulation.

8. The method of claim 5 in which an effective treatment amount of Product R is about 30 microliters per kilogram of body weight per day in a sterile injectable formulation for about one week, followed by about 15 microliters per kilogram of body weight per day in a sterile injectable formulation until said patient becomes asymptomatic or viral load becomes undetectable.

9. A method of treating a patient having antibodies to the FIV, comprising administering parenterally to said patient an effective FIV treatment amount of Product R in a sterile injectable formulation.

10. The method of claim 9 in which an effective treatment amount of Product R is in a range from about 5 microliters to about 40 microliters per kilogram of body weight per day in a sterile injectable formulation.

11. The method of claim 9 in which an effective treatment amount of Product R is in a range from about 10 microliters to about 25 microliters per kilogram of body weight per day in a sterile injectable formulation.

12. The method of claim 9 in which an effective treatment amount of Product R is about 30 microliters per kilogram of body weight per day in a sterile injectable formulation for about one week, followed by about 15 microliters per kilogram of body weight per day in a sterile injectable formulation until said patient becomes asymptomatic or viral load becomes undetectable.
